(19) United States
(12) Patent Application Publication
(10) Pub. No.: US 2020/0054541 A1
(43) Pub. Date: Feb. 20, 2020

(54) METHODS FOR REDUCING SWEAT AND/OR BODY ODOR, USING PHOSPHONATE COMPOUNDS WITH AMINE AND/OR HYDROXYL GROUPS

(71) Applicant: Henkel AG & Co. KGaA, Duesseldorf (DE)

(72) Inventors: BERNHARD BANOWSKI, Duesseldorf (DE); MARCUS CLAAS, Hilden (DE); DETLEF KELLER, Erkrath (DE)

(73) Assignee: Henkel AG & Co. KGaA, Duesseldorf (DE)

(21) Appl. No.: 16/609,710

(22) PCT Filed: May 4, 2018
(86) PCT No.: PCT/EP2018/061565

§ 371 Date: Oct. 30, 2019

(30) Foreign Application Priority Data
May 10, 2017 (DE) 10 2017 207 927.4

(51) Int. Cl. A61K 8/55; A61Q 15/00; A61K 8/02

(52) U.S. Cl. CPC A61K 8/55; A61K 8/0229; A61Q 15/00

(57) ABSTRACT

The present disclosure relates to a method for reducing transpiration of the body and/or reducing body odor triggered by the transpiration, in which an antiperspirant cosmetic agent (M1) which has a phosphonate compound having amine and/or hydroxyl groups is applied to the human skin and remains at the application site for at least about 1 hour. The use of the at least one phosphonate compound having amine and/or hydroxyl groups in these methods leads to an antiperspirant effect or to a reduction in body order triggered by the perspiration.

### CROSS-REFERENCE TO RELATED APPLICATION

This application is a U.S. National-Stage entry under 35 U.S.C. § 371 based on International Application No. PCT/EP2018/061565, filed May 4, 2018, which was published under PCT Article 21(2) and which claims priority to German Application No. 10 2017 207 927.4, filed May 10, 2017, which are all hereby incorporated in their entirety by reference.

### TECHNICAL FIELD

The present disclosure relates to a method for reducing transpiration of the body and/or reducing body odor triggered by the transpiration, in which an antiperspirant cosmetic agent (M1) containing phosphonate compounds having amine and/or hydroxyl groups is applied to the human skin and remains at the application site for at least about 1 hour. This antiperspirant agent (M1) contains at most about 1% by weight of antiperspirant aluminum and/or aluminum zirconium salts. The use of the phosphonate compound having amine and/or hydroxyl groups in the method as contemplated herein leads to an antiperspirant effect or to a reduction in body odor triggered by perspiration.

Furthermore, the present disclosure relates to a packaging unit (kit-of-parts), which in a first container (C1), comprises at least one phosphonate compound having amine and/or hydroxyl groups and at most about 1% by weight antiperspirant salts and in a second container (C2), comprises at least one antiperspirant compound.

Finally, the present disclosure relates to the use of at least one phosphonate compound having amine and/or hydroxyl groups for reducing the transpiration of the body and/or for reducing the body odor triggered by the transpiration.

### BACKGROUND

Washing, cleaning and caring for one's own body is a fundamental human need, and modern industry is constantly trying to meet these needs of mankind in a variety of ways. Especially important for daily hygiene is the prolonged elimination or at least reduction of body odor and underarm wetness. Numerous specific deodorant personal care antiperspirant products are known in the prior art which have been developed for use in body regions having a high density of sweat glands, in particular in the axillary region. They are packaged in different dosage forms, for example, as a powder, in stick form, as an aerosol spray, pump spray, liquid and gel roll-on application, cream, gel and as a soaked flexible substrate (deodorant wipes).

The cosmetic antiperspirants used to reduce transpiration in methods of the prior art contain at least one antiperspirant compound, in particular in the form of halides and/or hydroxy halides of aluminum and/or zirconium. These antiperspirant compounds reduce sweat secretion of the body by temporarily narrowing and/or obstructing the excretory ducts of the sweat glands, so that the amount of sweat can be reduced by from about 20 to about 60 percent. On the other hand, due to their antimicrobial effect, they prevent the degradation of initially odorless sweat to malodorous compounds and thus the formation of body odor.

However, the halides and/or hydroxy halides of aluminum and/or zirconium contained in the agents used can, in conjunction with the acidic pH of these agents, lead to unpleasant skin reactions in some users. In addition, the use of the previously mentioned antiperspirant compounds can lead to staining on the clothing.

There is therefore a need to replace the antiperspirant halides and/or hydroxy halides of aluminum and/or zirconium used with other antiperspirant cosmetic active substances in methods for reducing perspiration. These active substances should have a good antiperspirant effect, good skin compatibility and be easy to formulate. In addition, these antiperspirant active substances should have no negative impact on the storage stability of the cosmetic agents used.

### BRIEF SUMMARY

Packaging units and cosmetic methods are provided herein for reducing transpiration of the body and/or reducing body odor triggered by the transpiration. In accordance with an embodiment, a cosmetic method is provided in which an antiperspirant cosmetic is applied to the human skin and remains at the application site for at least about 1 hour. The antiperspirant cosmetic includes, in a cosmetically compatible carrier, based on the total weight of the cosmetic agent:

a) at least one phosphonate compound that includes at least one tertiary amine group and/or at least one secondary amine group and/or at least one primary amine group and/or at least one hydroxyl group, and

b) at most about 1% by weight of antiperspirant aluminum and/or aluminum zirconium salts.

In another embodiment, a packaging unit includes, assembled separately from one another,

a) at least one first container including cosmetic agent including, in a cosmetically compatible carrier, at least one phosphonate compound including at least one tertiary amine group and/or at least one secondary amine group and/or at least one primary amine group and/or at least one hydroxyl group and at most about 1% by weight of aluminum and/or aluminum zirconium antiperspirant salts based on the total weight of the cosmetic agent, and

b) at least one second container including a cosmetic agent comprising at least one antiperspirant active substance.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure has for its object to provide a method for reducing transpiration of the body and/or to reduce body odor triggered by the transpiration, which method avoids or at least mitigates the disadvantages of the prior art and which leads to a reliable reduction of underarm wetness and/or body odor with simultaneous good skin compatibility.

It has now surprisingly been found that the use of phosphonate compounds having at least one amine and/or hydroxyl group in cosmetic methods leads to an antiperspirant and/or odor-inhibiting effect, which is almost comparable with the achieved antiperspirant and odor-inhibiting effect of aluminum salts and/or aluminum zirconium salts used in methods of the prior art. However, no skin irritation is observed using these phosphonate compounds. Furthermore, these compounds can be incorporated stably into existing formulations and therefore do not lead to instabilities in storage of the cosmetic agents.

The subject of the present disclosure is thus a cosmetic method for reducing transpiration of the body and/or for reducing body odor triggered by the transpiration, in which an antiperspirant cosmetic agent (M1) is applied to the human skin and remains at the application site for at least about 1 hour, wherein the antiperspirant cosmetic agent (M1), in a cosmetically compatible carrier, based on the total weight of the cosmetic agent (M1),

a) contains at least one phosphonate compound containing at least one tertiary amine group and/or at least one secondary amine group and/or at least one primary amine group and/or at least one hydroxyl group, and
b) contains at most about 1% by weight of antiperspirant aluminum and/or aluminum zirconium salts.

An excellent reduction of sweat, in particular axillary sweat is achieved without skin irritation through the use of cosmetic agents which contain at least one phosphonate compound having at least one amine and/or hydroxyl group in the method as contemplated herein. Furthermore, a reduced formation of body odor is also observed through the use of the at least one phosphonate compound. An effective reduction of armpit sweat and/or body odor is thus ensured with the use of extremely small amounts of or in the absence of antiperspirant halides and/or hydroxy halides of aluminum and/or zirconium. In addition, these compounds can be incorporated without problems into existing formulations and do not lead to negative interactions with the other constituents of the cosmetic agents, thus achieving a high storage stability.

The term “antiperspirant” is understood as contemplated herein to mean decreasing or reducing the transpiration of the eccrine sweat glands of the body.

In addition, the term “aluminum and/or aluminum zirconium salts” in the context of the present disclosure is understood to mean in particular chlorides, bromides and iodides of aluminum and/or zirconium and compounds of the formulas Al(OH)_{y}X and Zr(OH)_{z}X, wherein X stands for a halide ion in the previously mentioned formulas.

In addition, the term “phosphonate compound” is understood to mean salts of organic compounds of phosphoric acid which have the general formula R—PO—(O⁻X⁺)₂. X⁺ here stands for a cation, for example H⁺, the radical R for an organic radical which has at least one group selected from primary, secondary and tertiary amine groups and also hydroxyl groups and combinations thereof. In addition, the radical R can also contain other phosphonic or phosphonate groups. However, the term “phosphonate compound” according to the present disclosure is not understood to mean phosphoric acid esters, in particular esters of phosphoric acid with organic radicals, of the formula R—O—PO—(O⁻X⁺)₂.

Finally, the term “fatty acid” in the context of the present disclosure is understood to mean aliphatic carboxylic acids which have unbranched or branched carbon radicals having 4 to 40 carbon atoms. The fatty acids used in the context of the present disclosure can be both naturally occurring and synthetically produced fatty acids. Furthermore, the fatty acids can be monounsaturated or polyunsaturated.

The statement % by weight relates in the present case, unless stated otherwise, to the total weight of the antiperspirant cosmetic agents (M1) used as contemplated herein without optional propellant present.

As contemplated herein, the antiperspirant cosmetic agent (M1) is advantageously used to reduce transpiration in the armpit and/or to reduce underarm odor. Preferred methods as contemplated herein are therefore exemplified in that the antiperspirant cosmetic agent (M1) is applied to the skin of the armpit.

The antiperspirant cosmetic agents (M1) used as contemplated herein contain the at least one phosphonate compound having at least one amine and/or hydroxyl group in a cosmetic carrier. As contemplated herein, this carrier is preferably anhydrous, aqueous, alcoholic or aqueous-alcoholic. The use of anhydrous carrier has proved to be advantageous in the case of powder-containing suspensions.

An anhydrous carrier is understood as contemplated herein to mean a carrier which contains less than 10% by weight of free water, based on the total weight of the antiperspirant cosmetic agent (M1). In the context of the present disclosure, free water is understood to mean water which is different from water of crystallization, water of hydration or similar molecularly bound water of the constituents used. The cosmetic carrier of the antiperspirant cosmetic agent (M1) used as contemplated herein preferably contains free water in a total amount of less than about 8.0% by weight, preferably less than about 5.0% by weight, more preferably less than about 3.0% by weight, yet more preferably less than about 1.0% by weight, in particular 0% by weight, based on the total weight of the antiperspirant cosmetic agent (M1).

Anhydrous carriers preferably contain a cosmetic oil which is liquid at 20° C. and 1013 hPa. These are understood to mean oils which are suitable for cosmetic use and which cannot be mixed with water in all amounts, which are neither fragrances nor essential oils. The at least one liquid cosmetic oil is preferably selected from the group of (i) volatile silicone oils, in particular volatile cyclic and linear silicone oils; (ii) volatile non-silicone oils, in particular liquid paraffin oils, isoparaffin oils, ester oils and organic alcohols; (iii) nonvolatile silicone oils; (iv) non-volatile non-silicone oils; and (v) mixtures thereof.

The term “volatile oil” refers to oils as contemplated herein which, at 20° C. and an ambient pressure of 1013 hPa, have a vapor pressure from about 2.66 Pa to about 40,000 Pa (from about 0.02 to about 300 mm Hg), preferably from about 10 to about 12,000 Pa (from about 0.1 to about 90 mm Hg), more preferably from about 13 to about 3000 Pa (from about 0.1 to about 23 mm Hg), in particular from about 15 to about 500 Pa (from about 0.1 to about 4 mm Hg).

In addition, the term “nonvolatile oils” in the context of the present disclosure is understood to mean cosmetic oils which have a vapor pressure of less than about 2.66 Pa (about 0.02 mm Hg) at 20° C. and an ambient pressure of 1013 hPa.

It can be preferred as contemplated herein to use mixtures of volatile silicone oils and volatile non-silicone oils as a carrier, since this achieves a drier skin feel. Furthermore, in the context of the present disclosure, it can be preferable when a nonvolatile silicone oil and/or a nonvolatile non-silicone oil is used as a carrier to mask insoluble constituents, such as talc, phosphonate compounds or ingredients dried on the skin.

The use of mixtures of the previously mentioned nonvolatile and volatile cosmetic oils is furthermore preferred as contemplated herein since parameters such as skin feel, visibility of the residue and stability of the cosmetic agent (M1) used as contemplated herein can be set and the agent can thus be better adapted to the needs of the consumer.

The cosmetic oil liquid at 20° C. and 1013 hPa is preferably used in a total amount of from about 1.0 to about 98% by weight, preferably from about 2.0 to about 85% by weight, more preferably from about 4.0 to about 75% by weight, yet more preferably from about 6.0 to about 70% by weight, still more preferably from about 8.0 to about 60% by weight, in particular from about 8.0 to about 20% by weight, based on the total weight of the antiperspirant cosmetic agent (M1).

In the context of the present disclosure, an aqueous carrier contains at least about 10% by weight of free water, based on the total weight of the antiperspirant cosmetic agent (M1). Preferably, the carrier contains free water in a total amount of from about 15 to about 98% by weight, preferably from about 30 to about 70% by weight, in particular from about 40 to about 60% by weight, based on the total weight of the antiperspirant cosmetic agent (M1).

As contemplated herein, alcoholic carriers contain at least about 1.0% by weight, based on the total weight of the antiperspirant cosmetic agent (M1), of a C₁-C₄ alcohol and/or of a C₂-C₆ alcohol having at least one hydroxyl group. These include, for example, ethanol, ethylene glycol, isopropanol, 1,2-propylene glycol, 1,3-propylene glycol, glycerol, n-butanol, 1,3-butylene glycol and mixtures thereof. Ethanol is particularly preferably used as the alcoholic carrier.

A preferred alcoholic carrier contains ethanol in a total amount of from about 1.0 to about 98% by weight, preferably from about 5.0 to about 70% by weight, more preferably from about 7.0 to about 50% by weight, in particular from about 10 to about 30% by weight, based on the total weight of the antiperspirant cosmetic agent (M1).

In the context of the present disclosure, aqueous-alcoholic carriers are understood to mean aqueous carriers which, in addition to water, additionally contain at least about 1.0% by weight of a C₁-C₄ alcohol and/or a C₂-C₆ alcohol containing at least one hydroxyl group, based on the total weight of the antiperspirant cosmetic agent (M1).

The antiperspirant and/or odor-inhibiting effect achieved by employing the method as contemplated herein is preferably achieved exclusively by using the at least one phosphonate compound. It is therefore advantageous within the context of the present disclosure when the cosmetic agent (M1) contains 0% by weight of antiperspirant aluminum and/or aluminum zirconium salts based on the total weight of the cosmetic agent (M1). Therefore, cosmetic agents (M1) used particularly preferably as contemplated herein contain no antiperspirant salts of aluminum and/or aluminum zirconium. In particular, none of the following antiperspirant salts of aluminum and/or aluminum zirconium are preferably present:

(i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate;
(ii) water-soluble astringent organic salts of aluminum, in particular aluminum chlorohydrex-propylene glycol, aluminum chlorohydrex-polyethylene glycol, aluminum-propylene glycol complexes, aluminum sesquichlorohydrex-propylene glycol, aluminum sesqui-chlorohydrex-polyethylene glycol, aluminum-propylene glycol-dichlorhydrex, aluminum-poly-ethylene glycol-dichlorhydrex, aluminum undecylenoyl collagen amino acid, sodium aluminum lactate, sodium aluminum chlorhydroxylactate, aluminum lipoamino acids, aluminum lactate, aluminum chlorohydroxyallantoinate, sodium aluminum chlorohydroxylactate;
(iii) water-soluble astringent inorganic aluminum zirconium salts, in particular aluminum zirconium trichlorohydrate, aluminum zirconium tetrachlorohydrate, aluminum zirconium pentachlorohydrate, aluminum zirconium octachlorohydrate;
(iv) water-soluble astringent organic aluminum zirconium salts, in particular aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; and
(v) mixtures thereof.

In the context of the present disclosure, it has proven advantageous to use phosphonate compounds which have at least one tertiary and/or secondary amine and/or at least one hydroxyl group in the molecule. Preferred methods as contemplated herein are therefore exemplified in that the at least one phosphonate compound contains at least one tertiary amine group and/or at least one secondary amine group and/or at least one hydroxyl group.

In a particularly preferred embodiment of the method as contemplated herein, phosphonate compounds having at least one tertiary or at least one secondary amine group are used. Preferred methods as contemplated herein are therefore exemplified in that the at least one phosphonate compound containing at least one tertiary amine group or at least one secondary amine group has the Formula (I)

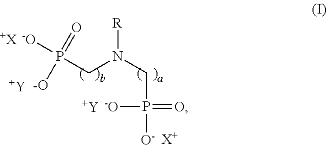

wherein
R stands for hydrogen, a *—(CH₂)ₓ—PO₃X⁺Y⁺ group with x=0 to about 4, a *—[CH₂—CH₂—NR¹]y-CH₂—CH₂—N(R¹)₂ group with y=0 to about 10 and R¹=*—CH₂—PO₃X⁺Y⁺ or a *—CH₂—CH₂—[NR²—CH₂—CH₂]_{z}—N(R³)₂ with z=1 to about 10, R²=*—(CH₂—CH₂—O)ₒ—H with o=1 to about 30 and R³=*—CH₂—PO₃X⁺Y⁺,
a and b, each independently of one another, stand for integers from 0 to about 10, preferably from 0 to about 6, more preferably from 0 to about 4, in particular 0 or 1,
X⁺ and Y⁺, each independently of one another, stand for a mono- or polyvalent cation from the group of alkali metals, alkaline earth metals, transition metals or organic cations, ammonium or hydrogen.

The term “organic cations” as contemplated herein are understood to mean cationic compounds which contain at least one carbon atom. The meaning of X⁺ and Y⁺ in the radicals R, R¹ and R³ here corresponds to the meaning of X⁺ and Y⁺ in the Formula (I). In addition, the attachment of the groups R, R¹ and R³ to the respective nitrogen atom is indicated by the symbol *. The use of these phosphonate compounds of the Formula (I) in the method as contemplated herein leads to a good antiperspirant effect, without negatively influencing the storage stability of the cosmetic agents or causing skin irritation.

In this context, it is preferred as contemplated herein when the organic cation is selected from the group of basic amino acids, choline, compounds of the formula N⁺(R₅)₄, wherein R₅ stands for linear or branched C₂-C₁₀ alkyl groups, which are optionally substituted with at least one hydroxyl group, imidazolium compounds, pyridinium compounds, pyrrolidinium compounds and sulfonium compounds. Basic amino acids have at least one proton acceptor in the side chain. Such amino acids are, for example, arginine, histidine and lysine. Compounds of the formula N⁺(R₅)₄ are understood to mean protonated alkylamines and alkanolamines. Particularly suitable alkylamines are monoalkyl, dialkyl and trialkylamines, such as diethyl and triethylamine, diisopropylamine and isopropylamine. Alkanolamines or amino alcohols have both an amino and a hydroxy group. The radical R₅ in the formula N⁺(R₅)₄ therefore contains at least one OH group. Alkanolamines which are suitable in the context of the present disclosure are, for example, choline, which is also known by the name [(2-hydroxyethyl) trimethylammonium and has the formula HO—CH₂—CH₂—N⁺(CH₃)₃. Further suitable alkanolamines are selected from the group of 2-aminoethane-1-ol (monoethanolamine), 3-aminopropan-1-ol, 4-aminobutan-1-ol, 5-aminopentan-1-ol, 1-aminopropane-2 ol (monoisopropanolamine), 1-aminobutan-2-ol, 1-aminopentan-2-ol, 1-aminopentan-3-ol, 1-aminopentan-4-ol, 2-amino-2-methyl-propanol, 2-amino 2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, triethanolamine, diethanolamine and triisopropanolamine. The imidazolium compounds (Formula (III)), pyridinium compounds (Formula (IV)), pyrrolidinium compounds (Formula (V)) and sulfonium compounds (Formula (VI)) which can be used in the context of the present disclosure preferably have the following formulas (III) to (VI), in which the radical R₆ in each case stands for hydrogen, OH or a linear or branched C₂-C₁₀ alkyl group which is optionally substituted by at least one hydroxyl group

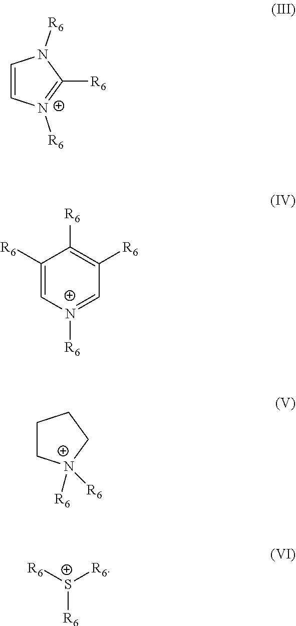

The mono- or polyvalent cation from the group of the alkali, alkaline earth and transition metals is preferably selected from certain representatives of these metals. Preferred methods as contemplated herein of this embodiment are therefore exemplified in that the mono- or polyvalent cation from the group of alkali metals, alkaline earth metals and transition metals is selected from Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺ or ¼ Ti⁴⁺, in particular Na⁺.

Within this embodiment, it is again preferred to use very specific phosphonate compounds of the Formula (I). It is therefore advantageous as contemplated herein when, in the Formula (I), a and b each stand for the integer 2, R stands for a *—(CH₂)ₓ—PO₃X⁺Y⁺ group with x=1 and X⁺ and Y⁺, each independently of one another, stand for hydrogen or Na⁺. This compound has the following structural formula and is also referred to as aminotrimethylene phosphonate (ATMP)

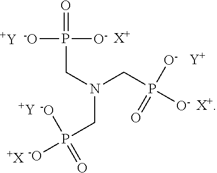

The use of this phosphonate compound leads to a significant antiperspirant effect, so that the use of further antiperspirant compounds, which are perceived by the consumer as critical or lead to skin irritation, can be dispensed with. In addition, this phosphonate compound can be incorporated stably into already existing cosmetic formulations and does not lead to negative interactions with other ingredients or to a reduced storage stability.

Instead of or in addition to the aminotrimethylene phosphonate (ATMP), it can also be provided to use phosphonate compounds with the designation ethylenediamine tetra (methylene phosphonate) (EDTMP). It can therefore also be advantageous as contemplated herein when, in the Formula (I), a and b each stand for the integer 1, R stands for a *—[CH₂—CH₂—NR¹]y-CH₂—CH₂—N(R¹)₂ group with y=0 and R¹=*—CH₂—PO₃X⁺Y⁺ and X⁺ and Y⁺, each independently of one another, stand for hydrogen or Na⁺. These compounds have the following structural formula and also lead to an excellent antiperspirant effect, but without adversely affecting the storage stability of the cosmetic agents (M1) or leading to undesirable skin irritation.

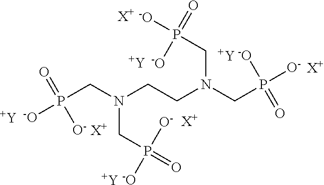

Instead of or in addition to the aminotrimethylene phosphonate (ATMP) and/or the ethylenediaminetetra (methylene phosphonate) (EDTMP), it can also be provided to use phosphonate compounds with the designation diethylenetriaminepenta (methylenephosphonate) (DTPMP). It can therefore also be advantageous as contemplated herein when, in the Formula (I), a and b each stand for the integer 1, R stands for a *—[CH₂—CH₂—NR¹]y-CH₂—CH₂—N(R¹)₂ group with y=1 and R¹=*—CH₂—PO₃X⁺Y⁺ and X⁺ and Y⁺, each independently of one another, stand for hydrogen or Na⁺. These compounds have the following structural formula and also lead to an excellent antiperspirant effect, but without adversely affecting the storage stability of the cosmetic agents (M1) or leading to undesirable skin irritation.

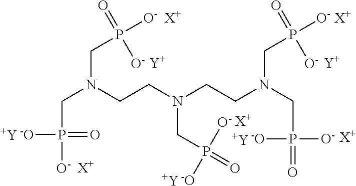

Instead of or in addition to the aminotrimethylene phosphonate (ATMP) and/or the ethylenediaminetetra (methylene phosphonate) (EDTMP) and/or the diethylene triamine penta (methylene phosphonate) (DTPMP), it can also be provided to use phosphonate compounds having the structural formula given below. It can therefore also be advantageous as contemplated herein when, in the Formula (I), a and b each stand for the integer 1, R stands for a *—CH₂—CH₂—[NR²—CH₂—CH₂]_{z}—N(R³)₂ with z=3 to 6, R²=*—(CH₂—CH₂—O)ₒ—H with o=2 to 6 and R³=*—CH₂—PO₃X⁺Y⁺ and X⁺ and Y⁺, each independently of one another, stand for hydrogen or Na⁺. These compounds also lead to an excellent antiperspirant effect, without however negatively influencing the storage stability of the cosmetic agents (M1) or leading to undesired skin irritation.

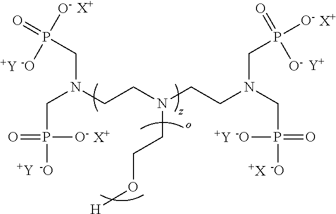

Instead of or in addition to the aminotrimethylene phosphonate (ATMP) and/or the ethylenediaminetetra (methylene phosphonate) (EDTMP) and/or the diethylene triamine penta (methylene phosphonate) (DTPMP) and/or compounds having the above structural formula, it can also be provided to use phosphonate compounds designated imidodiphosphate. It can therefore also be advantageous as contemplated herein when, in the Formula (I), a and b each stand for the integer 0, R stands for hydrogen and X⁺ and Y⁺, each independently of one another, stand for hydrogen or Na⁺. These compounds have the following structural formula and also lead to an excellent antiperspirant effect, but without adversely affecting the storage stability of the cosmetic agents (M1) or leading to undesirable skin irritation.

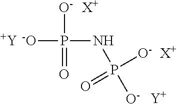

Within the context of a further, particularly preferred embodiment of the method as contemplated herein, phosphonate compounds having at least one hydroxyl group are used. Preferred methods as contemplated herein are therefore exemplified in that the at least one phosphonate compound which contains at least one hydroxyl group has the Formula (II)

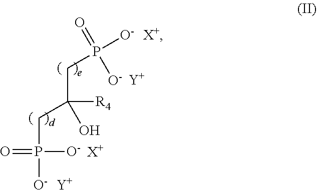

wherein
R₄ stands for a linear or branched, saturated or unsaturated C₁-C₃₀ alkyl group, d and e, each independently of one another, stand for integers from 0 to about 10, preferably from 0 to about 6, more preferably from 0 to about 4, in particular 0 or 1, X⁺ and Y⁺, in each case independently of each other, stand for a mono- or polyvalent cation from the group of alkali metals, alkaline earth metals, transition metals or organic cations, ammonium or hydrogen.

The term “organic cations” as contemplated herein are understood to mean cationic compounds which contain at least one carbon atom. The use of these phosphonate compounds of the Formula (II) in the method as contemplated herein leads to a good antiperspirant effect, without negatively influencing the storage stability of the cosmetic agents or causing skin irritation.

In this context, it is preferred as contemplated herein when the organic cation is selected from the group of basic amino acids, choline, compounds of the formula N⁺(R₅)₄, wherein R₅ stands for linear or branched C₂-C₁₀ alkyl groups, which are optionally substituted with at least one hydroxyl group, imidazolium compounds, pyridinium compounds, pyrrolidinium compounds and sulfonium compounds. Basic amino acids have at least one proton acceptor in the side chain. Such amino acids are, for example, arginine, histidine and lysine. Compounds of the formula N⁺(R₅)₄ are understood to mean protonated alkylamines and alkanolamines. Particularly suitable alkylamines are monoalkyl, dialkyl and trialkylamines, such as diethyl and triethylamine, diisopropylamine and isopropylamine. Alkanolamines or amino alcohols have both an amino and a hydroxy group. The radical R₅ in the formula N⁺(R₅)₄ therefore contains at least one OH group. Alkanolamines which are suitable in the context of the present disclosure are, for example, choline, which is also known by the name [(2-hydroxyethyl) trimethylammonium and has the formula HO—CH₂—CH₂—N⁺(CH₃)₃. Further suitable alkanolamines are selected from the group of 2-aminoethane-1-ol (monoethanolamine), 3-aminopropan-1-ol, 4-aminobutan-1-ol, 5-aminopentan-1-ol, 1-aminopropane-2 ol (monoisopropanolamine), 1-aminobutan-2-ol, 1-aminopentan-2-ol, 1-aminopentan-3-ol, 1-aminopentan-4-ol, 2-amino-2-methyl-propanol, 2-amino 2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, triethanolamine, diethanolamine and triisopropanolamine. The imidazolium compounds (Formula (III)), pyridinium compounds (Formula (IV)), pyrrolidinium compounds (Formula (V)) and sulfonium compounds (Formula (VI)) which can be used in the context of the present disclosure preferably have the following formulas (III) to (VI), in which the radical R₆ in each case stands for hydrogen, OH or a linear or branched C₂-C₁₀ alkyl group which is optionally substituted by at least one hydroxyl group

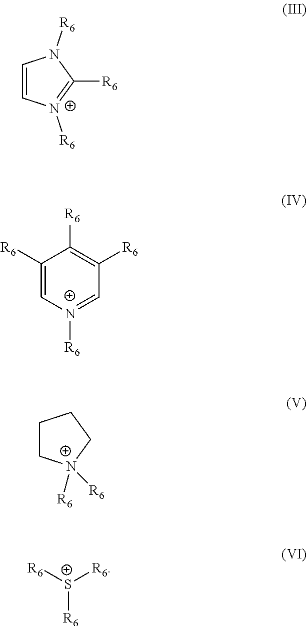

The mono- or polyvalent cation from the group of the alkali, alkaline earth and transition metals is preferably selected from certain representatives of these metals. Preferred methods d herein of this embodiment are therefore exemplified in that the mono- or polyvalent cation from the group of alkali metals, alkaline earth metals and transition metals is selected from Na⁺, K⁺, Li⁺, ½ Mg²⁺, ½ Ca²⁺, ½ Zn²⁺, ½ Mn²⁺, ½ Cu²⁺, ⅓ Fe³⁺, ¼ Zr⁴⁺ or ¼ Ti⁴⁺, in particular Na⁺.

Within this embodiment, it is again preferred to use very specific phosphonate compounds of the Formula (I). It is therefore advantageous as contemplated herein when, in the Formula (II), R₄ stands for a C₁₁ alkyl group, d and e each stand for the integer 0 and X⁺ and Y⁺, each independently of one another, stand for hydrogen or Na⁺. This compound has the following structural formula and is also referred to as (1-hydroxy-1,1-dodecanediyl) bis (phosphonate).

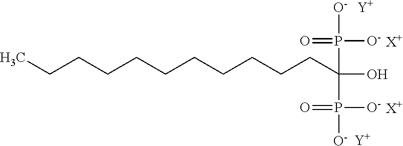

The use of this phosphonate compound leads to an excellent antiperspirant effect, so that the use of further antiperspirant compounds, which are perceived by the consumer as critical or lead to skin irritation, can be dispensed with. In addition, this phosphonate compound can be incorporated stably into already existing cosmetic formulations and does not lead to negative interactions with other ingredients or to a reduced storage stability.

In the context of the present disclosure, it is advantageous to use the at least one phosphonate compound in the cosmetic agent (M1) in specific quantitative ranges. Methods preferred as contemplated herein are therefore exemplified in that the cosmetic agent (M1) contains at least one phosphonate compound a) in a total amount of from about 0.1 to about 40% by weight, preferably from about 0.2 to about 30% by weight, more preferably from about 1.0 to about 25% by weight, in particular from about 2.0 to about 10% by weight, based on the total weight of the cosmetic agent (M1). The use of the at least one phosphonate compound, in particular of the previously mentioned specific phosphonate compounds, in the previously mentioned total amounts in the method as contemplated herein leads to an excellent reduction in transpiration and/or body odor triggered by the transpiration. Furthermore, no skin irritation or storage instabilities occur when using the at least one phosphonate compound in the previously mentioned amounts. If a mixture of different phosphonate compounds is used, the quantities given above refer to the total amount of the mixture of phosphonate compounds.

The antiperspirant cosmetic agents (M1) used in the method as contemplated herein preferably have a certain pH value. Within this range, a stable formulation of the antiperspirant cosmetic agents (M1) used as contemplated herein is possible without the occurrence of undesirable interactions between the ingredients. Furthermore, no skin irritation occurs when these agents are used at these pH values. It is therefore preferred when the cosmetic agent (M1) has a pH value of from about pH 2.5 to about pH 9.0, preferably of from about pH 2.5 to about pH 8.5, more preferably of from about pH 2.5 to about pH 8.0, in particular of from about pH 3.0 to about pH 7.0. The desired pH value can be set by using acids and bases which are known to the person skilled in the art and are customary in antiperspirant cosmetic agents.

The antiperspirant cosmetic agent (M1) used in the method as contemplated herein can contain, in addition to the previously mentioned ingredients, further substances.

As contemplated herein, the antiperspirant cosmetic agent (M1) additionally preferably contains at least one wide adjuvant selected from the group of (i) emulsifiers and/or surfactants; (ii) thickening agents; (iii) chelating agents; (iv) deodorant active substances; (v) polyethylene glycols; (vi) skin-cooling active substances; (vii) pH adjusters; (viii) skin care active substances, such as moisturizing substances, skin soothing substances, skin lightening substances, skin smoothing substances; (ix) waxes; (x) preservatives; (xi) plant extracts; (xii) absorbers; (xiii) perfume oils; and (xiv) mixtures thereof.

Emulsifiers and surfactants which are preferably suitable as contemplated herein are selected from anionic, cationic, nonionic, amphoteric, in particular ampholytic and zwitterionic emulsifiers and surfactants. Surfactants are amphiphilic (bifunctional) compounds which include at least one hydrophobic and at least one hydrophilic molecule part. The hydrophobic radical is preferably a hydrocarbon chain having from about 8 to about 28 carbon atoms, which can be saturated or unsaturated, linear or branched. Particularly preferably, this C₈-C₂₈ alkyl chain is linear. Emulsifiers and surfactants which can preferably be used in the context of the present disclosure are disclosed, for example, in the published patent application DE 10 2012 222 692 A1.

Substances which are selected from cellulose ethers, xanthan gum, sclerotium gum, succinoglucans, polygalactomannans, pectins, agar, carrageenan, tragacanth, gum arabic, karaya gum, tara gum, gellan, gelatin, propylene glycol alginate, alginic acids and salts thereof, polyvinylpyrrolidones, polyvinyl alcohols, polyacrylamides, physically (for example, by pre-gelatinization) and/or chemically modified starches, acrylic acid acrylate copolymers, acrylic acid acrylamide copolymers, acrylic acid vinylpyrrolidone copolymers, acrylic acid vinyl formamide copolymers and polyacrylates are preferably used to thicken the antiperspirant cosmetic agents as contemplated herein. Cellulose ethers, such as carboxyalkylcelluloses, are particularly preferably used as thickening agents. Particularly preferred thickening agents are furthermore selected from carbomers. Carbomers are thickening crosslinked polymers of acrylic acid, methacrylic acid and salts thereof. The crosslinking can be carried out by employing polyfunctional compounds such as polyalkylene ethers of polysaccharides or polyalcohols, for example, sucrose allyl ether, pentaerythritol allyl ether, propylene allyl ether. Preferred in the context of the present disclosure are homopolymers of acrylic acid or salts thereof, which are crosslinked with a pentaerythritol allyl ether, a sucroseallyl ether or a propylene allyl ether. A thickening agent which can be used in the context of the present disclosure is a copolymer of C₁₀₋₃₀ alkyl acrylate, acrylic acid, methacrylic acid and esters thereof, which is crosslinked with a sucrose allyl ether or a pentaerythritol allyl ether. Thickening agents based on carbomer are the products available under the trade name Carbopol® (BF Goodrich, Ohio, USA) such as Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21.

Furthermore, lipophilic thickening agents can be used to thicken the antiperspirant cosmetic agents (M1) used as contemplated herein. Lipophilic thickening agents preferred as contemplated herein are selected from hydrophobized clay minerals, bentonites, hectorites, fumed silicas and their derivatives.

As an additional adjuvant, the antiperspirant cosmetic agents (M1) used as contemplated herein can contain at least one chelating agent, in a total amount of from about 0.01 to about 3.0% by weight, preferably from about 0.02 to about 1.0% by weight, in particular from about 0.05 to about 0.1% by weight, based on the total weight of the antiperspirant agent (M1). In the context of the present disclosure, preferred chelating agents are selected from the group of β-alaninediacetic acid, cyclodextrin, sodium, potassium, calcium disodium, ammonium and triethanolamine salts of ethylenediaminetetraacetic acid (EDTA), hydroxyethylethylenediaminetetraacetic acid (HEDTA) and their sodium salts, sodium salts of nitrilotriacetic acid (NTA), diethylenetriaminepentaacetic acid, phytic acid, hydroxypropyl cyclodextrin, methylcyclodextrin, diethylenetriamine pentaacetate pentasodium, pentasodium triphosphate, sodium hydroxymethylglycinate, sodium phytate, sodium polydimethylglycinophenol sulfonate, tetrahydroxyethylethylenediamine, tetrahydroxypropylethylenediamine, tetrapotassium etidronate, tetrasodium etidronate, tetrasodium iminodisuccinate, trisodium ethylenediamine disuccinate, tetrasodium N,N-bis glutamate (carboxymethyl), tetrasodium DL-alanine-N,N-diacetate and desferrioxamine.

The deodorizing effect of the antiperspirant cosmetic agents (M1) used as contemplated herein can be further increased when at least one deodorant active substance having antibacterial and/or bacteriostatic and/or enzyme inhibiting and/or odor-neutralizing and/or odor-absorbing action is additionally present in a total amount of from about 0.0001 to about 40% by weight, preferably from about 0.2 to about 20% by weight, more preferably from about 1 to about 15% by weight, in particular from about 1.5 to about 5.0% by weight, based on the total weight of the antiperspirant cosmetic agent (M1). If ethanol is used in the agents as contemplated herein, this does not apply in the context of the present disclosure as a deodorant active substance, but rather as a constituent of the carrier.

Preferred antiperspirant cosmetic agents (M1) used as contemplated herein can further contain at least one water-soluble polyethylene glycol having from about 3 to about 50 ethylene oxide units.

In addition, the antiperspirant cosmetic agents (M1) used as contemplated herein can further contain at least one skin-cooling active substance. Skin-cooling active substances suitable as contemplated herein are, for example, menthol, isopulegol and menthol derivatives, for example, menthyl lactate, menthyl glycolate, menthyl ethyl oxamate, menthyl pyrrolidone carboxylic acid, menthyl methyl ether, menthoxypropanediol, menthone glycerol acetal (9-methyl-6-(1-methylethyl)-1,4-dioxaspiro (4.5) decane-2-methanol), monomethyl succinate, 2-hydroxymethyl-3,5,5-trimethylcyclohexanol and 5-methyl-2-(1-methylethyl) cyclohexyl-N-ethyloxamate. Preferred skin-cooling active ingredients are menthol, isopulegol, menthyl lactate, menthoxypropanediol, menthylpyrrolidonecarboxylic acid and 5-methyl-2-(1-methylethyl) cyclohexyl-N-ethyl oxamate, and mixtures of these substances, in particular mixtures of menthol and menthyl lactate, menthol, menthol glycolate and menthyl lactate, menthol and menthoxypropanediol or menthol and isopulegol.

As contemplated herein, acids and/or alkalizing agents and/or buffers are preferably used as pH-adjusting agents. As contemplated herein, inorganic acids (such as hydrochloric acid, sulfuric acid or phosphoric acid) or organic acids (such as lactic acid, citric acid, tartaric acid or malic acid) are preferably used as acids. Alkaliating agents which can be used as contemplated herein are preferably selected from the group which is formed from ammonia, basic amino acids, alkali metal hydroxides, alkaline earth metal hydroxides, carbonates and bicarbonates, alkanolamines, for example amino-2-methyl-1-propanol, monoethanolamine, triethanolamine, diethanolamine and triisopropanolamine, alkali metal metasilicates, urea, morpholine, N-methylglucamine, imidazole. Lithium, sodium, potassium, in particular sodium or potassium are preferably used as alkali metal ions. Suitable buffer systems in the context of the present disclosure are, in particular, carbonic acid bicarbonate buffers, carbonic acid/silicate buffers, acetic acid/acetate buffers, ammonia buffers, citric acid or citrate buffers, buffers based on tris (hydroxymethyl) aminomethane, buffers based on 4-(2-hydroxyethyl)-1-piperazineethanesulfonic acid, buffer based on 4-(2-hydroxyethyl)-piperazine-1-propanesulfonic acid, buffer based on 2-(N-morpholino) ethanesulfonic acid and barbital acetate buffer. The choice of the corresponding buffer system depends on the desired pH value of the antiperspirant cosmetic agents (M1) used as contemplated herein. However, the at least one phosphonate compound used as contemplated herein does not fall under the previously mentioned pH adjusting agents in the form of acids and buffer systems.

Furthermore, the antiperspirant cosmetic agents (M1) used as contemplated herein can contain at least one wax. This term is understood to mean substances which are kneadable at 20° C. or solid to brittle, have a coarse to finely crystalline structure and are translucent to opaque in color but not glassy. Furthermore, these substances melt above 25° C. without decomposition, are slightly liquid (less viscous) above the melting point, have a strong temperature-dependent consistency and solubility and are polishable under light pressure. Preferably, this wax is selected from the group of (i) fatty acid glycerol mono-, di- and triesters; (ii) Butyrospermum Parkii (shea butter); (iii) esters of saturated monovalent C₈₋₁₈ alcohols with saturated C₁₂₋₁₈ monocarboxylic acids; (iv) linear primary C₁₂-C₂₄ alkanols; (v) esters of a saturated monovalent C₁₆₋₆₀ alkanol and a saturated C₈-C₃₆ monocarboxylic acid; (vi) glycerol triesters of saturated linear C₁₂₋₃₀ carboxylic acids, which can be hydroxylated, such as glycerol esters of hydrogenated vegetable oils; (vii) natural vegetable waxes; (viii) animal waxes; (ix) synthetic waxes; and (x) mixtures thereof.

The wax is preferably used in a total amount of from about 0.01 to about 60% by weight, preferably from about 3.0 to about 40% by weight, more preferably from about 5.0 to about 30% by weight, particularly preferably from about 6.0 to about 25% by weight, based on the total weight of the antiperspirant cosmetic agent (M1).

As contemplated herein, it is further preferred when the antiperspirant cosmetic agent (M1) used as contemplated herein additionally contains at least one preservative. Preservatives which are preferred as contemplated herein are formaldehyde releasers iodopropynyl butylcarbamates, parabens, phenoxyethanol, ethanol, benzoic acid and salts thereof, dibromodicyanobutane, 2-bromo-2-nitro-propane-1,3-diol, imidazolidinyl urea, 5-chloro-2-methyl-4-isothiazolin-3-one, 2-chloroacetamide, benzalkonium chloride, benzyl alcohol, salicylic acid and salicylates. Further preservatives which can be used in the context of the present disclosure are cosmetic raw materials having preserving properties or raw materials which support or enhance the preservative action of the previously mentioned preservatives. The preservatives are preferably present in a total amount of from about 0.01 to about 10% by weight, preferably from about 0.1 to about 7.0% by weight, more preferably from about 0.2 to about 5.0% by weight, in particular from about 0.3 to about 2.0% by weight, based on the total weight of the antiperspirant cosmetic agent (M1).

Antiperspirant cosmetic agents (M1) used as contemplated herein can furthermore contain at least one plant extract. Plant extracts are usually prepared by extraction of the entire plant, but in some cases also exclusively from flowers and/or leaves and/or seeds and/or other parts of plants. Especially preferred as contemplated herein are the extracts of aloe vera, green tea, witch hazel, bamboo, chamomile, marigold, pansy, peony, horse chestnut, sage, willow bark, cinnamon tree, chrysanthemums, oak bark, stinging nettle, hops, burdock root, horsetail, hawthorn, lime blossom, almonds, spruce needles, sandalwood, juniper, coconut, kiwi, guava, lime, mango, apricot, wheat, melon, orange, grapefruit, avocado, rosemary, birch, beech shoots, mallow, meadowfoam, yarrow, quender, thyme, melissa, hominy, marshmallow (Althaea), mallow (Malva sylvestris), violets, blackcurrant leaves, coltsfoot, cinquefoil, ginseng, ginger root and sweet potato. Furthermore, algae extracts can also be used. These are preferably derived from green algae, brown algae, red algae or blue-green algae (cyanobacteria). Advantageous algae extracts come from kelp, blue-green algae, from the green alga Codium tomentosum and from the brown algae Fucus vesiculosus. A particularly preferred algae extract is derived from blue-green algae of the species Spirulina, which were cultured in a magnesium-enriched medium. The at least one plant extract is preferably present in total amounts of from about 0.01 to about 5.0% by weight, preferably from about 0.1 to about 2.0% by weight, in particular from about 0.5 to about 1.0% by weight, each based on the total weight of the antiperspirant agent (M1).

It can further be preferred as contemplated herein when the antiperspirant cosmetic agents (M1) contain at least one absorber. Preferred absorbers are selected from silicates. These can also simultaneously support the rheological properties of antiperspirant agents (M1). Among the silicates which are particularly advantageous as contemplated herein are, especially, phyllosilicates and, among these, in particular montmorillonite, kaolinite, illite, beidellite, nontronite, saponite, hectorite, bentonite, smectite and talc. Further preferred absorbers are, for example, zeolites, zinc ricinoleate, cyclodextrins, certain metal oxides, such as alumina, and chlorophyll. They are preferably used in a total amount of from about 0.1 to about 10% by weight, preferably from about 0.5 to about 7.0% by weight, in particular from about 1.0 to about 5.0% by weight, each based on the total weight of antiperspirant agent (M1).

In the context of the present disclosure, it is preferred when the antiperspirant cosmetic agent (M1) used as contemplated herein is present as a water-in-oil emulsion. This can in particular be a sprayable water-in-oil emulsion which can be sprayed by employing a propellant. If the antiperspirant cosmetic agents (M1) used as contemplated herein contain a propellant, this is preferably present in a total amount of from about 1 to about 98% by weight, preferably from about 20 to about 90% by weight, preferably from about 30 to about 85% by weight, in particular from about 40 to about 75% by weight, based on the total weight of the antiperspirant cosmetic agent (M1). The total weight of the agent (M1) here is the sum of the weight of all constituents of the agent including the propellant. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes, both individually and in mixtures thereof. Hydrophilic propellant gases, such as carbon dioxide, can also be used advantageously in the context of the present disclosure, when the proportion of hydrophilic gases is selected low and lipophilic propellant gas (for example, propane/butane) is present in excess. Particularly preferred are propane, n-butane, isobutane and mixtures of these propellant gases. It has been found that the use of n-butane as the only propellant gas as contemplated herein can be particularly preferred.

However, it can equally be preferred as contemplated herein when the antiperspirant cosmetic agent (M1) used as contemplated herein is present as an oil-in-water emulsion. In this case, the cosmetic agent as contemplated herein is preferably sprayed as a propellant-free pump spray or squeeze spray or applied as a roll-on.

The application of the antiperspirant cosmetic agent (M1) used as contemplated herein can be carried out by employing various methods. According to a preferred embodiment, the antiperspirant cosmetic agent used (M1) as contemplated herein is formulated as a spray application. The spray application is carried out with a spray device which contains a filling of the liquid, viscous-flowable, suspension-formed or powdered antiperspirant cosmetic agent (M1) as contemplated herein in a container. The filling can be under the pressure of a propellant as described previously (compressed gas cans, compressed gas packages, aerosol packages), or it can be a mechanically operated pumping atomizer without propellant gas (pump sprays/squeeze bottle). The atomization of the antiperspirant cosmetic agent (M1) used in the method as contemplated herein can be carried out physically, mechanically or electromechanically, for example, by piezoelectric effects or electrical pumps. The at least one phosphonate compound in these sprayable, suspension-type cosmetic agents (M1) preferably has an average particle size D₅₀ of from about 1 to about 300 μm, preferably from about 5 to about 125 μm, in particular from about 10 to about 100 μm.

The antiperspirant cosmetic agent (M1) used as contemplated herein can furthermore be preferably formulated as a stick, soft solid, cream, gel, roll-on, loose or compact powder. The formulation of the antiperspirant cosmetic agents (M1) used as contemplated herein in a particular administration form, such as, for example, an antiperspirant roll-on, an antiperspirant stick or an antiperspirant gel, is preferably based on the requirements of the intended use. Depending on the intended use, the antiperspirant cosmetic agents (M1) used as contemplated herein can therefore be present in solid, semisolid, liquid, disperse, emulsified, suspended, gelatinous, multiphase or powdery form. In the context of the present disclosure, the term liquid also includes any types of solids dispersions in liquids. Furthermore, multiphase antiperspirant cosmetic agents (M1) used as contemplated herein in the context of the present disclosure are understood to mean agents which have at least two different phases with a phase separation and in which the phases can be arranged horizontally, one above the other, or vertically, that is next to each other. The application can be carried out, for example, using a roller ball applicator, a pump sprayer or by employing a solid stick. If the antiperspirant cosmetic agents (M1) used as contemplated herein are in the form of solid sticks, it is preferred when the at least one phosphonate compound has an average particle size D₅₀ of from about 1 to about 300 μm, preferably from about 5 to about 125 μm, preferably from about 10 to about 100 μm, in particular from about 3 to about 20 μm.

It can also be preferred in the context of the present disclosure when the antiperspirant cosmetic agent (M1) is contained on and/or in a disposable substrate selected from the group of wipes, pads and dabbers. Particularly preferred are wet wipes, that is, wet wipes prefabricated for the user, preferably individually packaged, as they are, for example, well known from the field of glass cleaning or from the field of wet toilet paper. Such wet wipes, which can advantageously also contain preservatives, are impregnated or applied with an antiperspirant cosmetic agent (M1) used as contemplated herein and preferably individually packaged. Preferred substrate materials are selected from porous sheet-like cloths. These wipes include wipes made of woven and nonwoven synthetic and natural fibers, felt, paper or foam, such as hydrophilic polyurethane foam. Deodorizing or antiperspirant substrates which are preferred as contemplated herein can be obtained by saturating or impregnating or also by fusing an antiperspirant cosmetic agent (M1) used as contemplated herein onto a substrate.

In the following tables, particularly preferred embodiments AF 1 to AF 44 of the cosmetic agents (M1) used in the method as contemplated herein are listed (all data specified in % by weight, unless stated otherwise):

| | AF 1 | AF 2 | AF 3 | AF 4 |
|---|---|---|---|---|
| Phosphonate compound a)¹⁾ | 0.1-40 | 0.2-30 | 1.0-25 | 2.0-10 |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 5 | AF 6 | AF 7 | AF 8 |
| Phosphonate compound a) of the Formula | 0.1-40 | 0.2-30 | 1.0-25 | 2.0-10 |
| (I) | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 9 | AF 10 | AF 11 | AF 12 |
| Phosphonate compound a) of the Formula | 0.1-40 | 0.2-30 | 1.0-25 | 2.0-10 |
| (I)³⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 13 | AF 14 | AF 15 | AF 16 |
| Phosphonate compound a) of the Formula | 0.1-40 | 0.2-30 | 1.0-25 | 2.0-10 |
| (I)⁴⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 17 | AF 18 | AF 19 | AF20 |
| Phosphonate compound a) of the Formula | 0.1-40 | 0.2-30 | 1.0-25 | 2.0-10 |
| (I)⁵⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 21 | AF 22 | AF 23 | AF 24 |
| Phosphonate compound a) of the Formula | 0.1-40 | 0.2-30 | 1.0-25 | 2.0-10 |
| (I)⁶⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 25 | AF 26 | AF 27 | AF 28 |
| Phosphonate compound a) of the Formula | 0.1-40 | 0.2-30 | 1.0-25 | 2.0-10 |
| (I)⁷⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 29 | AF 30 | AF 31 | AF 32 |
| Phosphonate compound a) of the Formula | 0.1-40 | 0.2-30 | 1.0-25 | 2.0-10 |
| (II) | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 33 | AF 34 | AF 35 | AF 36 |
| Phosphonate compound a) of the Formula | 0.1-40 | 0.2-30 | 1.0-25 | 2.0-10 |
| (I)⁸⁾ | | | | |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 37 | AF 38 | AF 39 | AF 40 |
| Phosphonate compound a)⁹⁾ | 0.1-40 | 0.2-30 | 1.0-25 | 2.0-10 |
| Aluminum and/or aluminum zirconium salt | 0-1.0 | 0-0.5 | 0-0.2 | 0 |
| Cosmetic carrier²⁾ | Ad 100 | Ad 100 | Ad 100 | Ad 100 |
| | AF 41 | AF 42 | AF 43 | AF 44 |
| Phosphonate compound a) of the Formula | 0.01-40 | 0.2-30 | 1.0-25 | 2.0-20 |
| (I)³⁾ | | | | |
| Aluminum and/or aluminum zirconium salt | 0-1.0 | 0-0.5 | 0-0.2 | 0 |
| Emulsifier¹⁰⁾ | 0.1-15 | 0.5-10 | 1.0-8.0 | 2.0-6.0 |
| Cosmetic carrier²⁾ | Ad 100 | Ad 100 | Ad 100 | Ad 100 |

¹⁾Phosphonate compound contains at least one tertiary amine group and/or at least one secondary amine group and/or at least one hydroxyl group
²⁾anhydrous, aqueous or aqueous-alcoholic carrier,
³⁾a = b = 1, R = *—(CH₂)ₓ—PO₃X⁺Y⁺ group with x = 1 and X⁺ and Y⁺ each independently of one another stand for hydrogen or Na⁺,
⁴⁾a = b = 1, R = *—[CH₂—CH₂—NR¹]_{y}—CH₂—CH₂—N(R¹)₂ group with y = 0 and R¹ = *—CH₂—PO₃X⁺Y⁺, X⁺ and Y⁺ each independently of one another stand for hydrogen or Na⁺,
⁵⁾a = b = 1, R = *—[CH₂—CH₂—NR¹]_{y}—CH₂—CH₂—N(R¹)₂ group with y = 1 and R¹ = *—CH₂—PO₃X⁺Y⁺, X⁺ and Y⁺ each independently of one another stand for hydrogen or Na⁺,
⁶⁾a = b = 1, R = *—CH₂—CH₂—[NR²—CH₂—CH₂]_{z}—N(R³)₂ with z = 3 to 6, R² = *—(CH₂—CH₂—O)ₒ—H with o = 2 to 6 and R³ = *—CH₂—PO₃X⁺Y⁺, X⁺ and Y⁺ each independently of one another stand for hydrogen or Na⁺,
⁷⁾a = b = 0, R = hydrogen and X⁺ and Y⁺ each independently of one another stand for hydrogen or Na⁺,
⁸⁾R₄ = C₁₁ alkyl group, d = e = 0 and X⁺ and Y⁺ each independently of one another stand for hydrogen or Na⁺,
⁹⁾mixture of at least two phosphonate compounds of the Formula (I) or mixture of at least one phosphonate compound of the Formula (I) and of the Formula (II),
¹⁰⁾emulsifier selected from the group of ethoxylated C₁₂-C₁₈ alkanols with from about 10 to about 30 moles of ethylene oxide per mole of alkanol, ethoxylated C₈-C₂₄ carboxylic acids with from about 10 to about 30 moles of ethylene oxide per mole of carboxylic acid, C₈-C₂₂ alkyl mono- and oligoglycosides, ethoxylated sterols having a degree of ethoxylation of more than about 5, partial esters of poly glycerols with from about 2 to about 10 glycerol units and from about 1 to about 4 saturated or unsaturated, linear or branched, optionally hydroxylated C₈-C₃₀ carboxylic acid radicals, in particular ethoxylated C₁₂-C₁₈ alkanols with from about 10 to about 30 moles of ethylene oxide per mole of alkanol.

The use of the above-mentioned embodiments AF1 to AF44 in the method as contemplated herein leads to a good antiperspirant and/or odor-inhibiting effect, however without causing skin irritation. Furthermore, these embodiments have a high storage stability.

In the context of the present disclosure, it can also be provided to apply a further cosmetic agent (M2) which contains at least one antiperspirant active substance. The agents (M1) and (M2) can be applied in any order one after the other or simultaneously. In this case, it can be provided, for example, to first apply the cosmetic agent (M1) and then the cosmetic agent (M2). However, it is also possible to first apply the cosmetic agent (M2) and then the cosmetic agent (M1). In addition, the cosmetic agent (M1) and the cosmetic agent (M2) can be applied simultaneously. The time span between the application of the two agents (M1) and (M2) is preferably from 0 seconds (simultaneous application) to about 24 hours.

It is therefore preferred in the context of the present disclosure when a further cosmetic agent (M2), containing at least one antiperspirant aluminum and/or aluminum zirconium salt in a cosmetically compatible carrier is applied to the skin. The cosmetic agent (M2) here is different from the cosmetic agent (M1) and contains no phosphonate compounds having amine and/or hydroxyl groups. The antiperspirant effect of the method as contemplated herein can be further increased in this way.

If a further cosmetic agent (M2) is to be used in the method as contemplated herein, it is advantageous to store the individual cosmetic agents (M1) and (M2) in respectively separate containers.

A further subject of the present disclosure is therefore a packaging unit (kit-of-parts), comprising, assembled separately from one another,

a) at least one first container (C1) containing a cosmetic agent (M1) comprising, in a cosmetically compatible carrier, at least one phosphonate compound containing at least one tertiary amine group and/or at least one secondary amine group and/or at least one primary amine group and/or at least one hydroxyl group and at most about 1% by weight of aluminum and/or aluminum zirconium antiperspirant salts based on the total weight of the cosmetic agent (M1), and
b) at least one second container (C2) containing a cosmetic agent (M2) comprising at least one antiperspirant active substance.

As contemplated herein, the term “antiperspirant active substance” is understood to mean active substances which decrease or reduce the transpiration of the eccrine sweat glands of the body. The antiperspirant active substance in the cosmetic agent (M2) is different from the phosphonate compound contained in the agent (M1). The cosmetic agents (M2) therefore contain no phosphonate compounds having amine and/or hydroxyl groups. In the context of the present disclosure, the antiperspirant aluminum and/or aluminum zirconium salts mentioned in connection with the first subject of the present disclosure are preferably used as antiperspirant active substance in the cosmetic agent (M2).

Statements made about the cosmetic agents (M1) used in the method as contemplated herein apply mutatis mutandis with respect to the cosmetic agent (M1) in the container (C1), in particular with regard to the phosphonate compound, and with regard to the other constituents of the agent (M2).

Finally, a further subject of the present disclosure is the use of at least one phosphonate compound containing at least one tertiary amine group and/or at least one secondary amine group and/or at least one primary amine group and/or at least one hydroxyl group to reduce the transpiration of the body and/or to reduce the body odor triggered by the transpiration.

The statements made on the method as contemplated herein, in particular on the agent (M1) used there, and on the packaging unit as contemplated herein, apply mutatis mutandis with regard to the phosphonate compound used as contemplated herein.

The following examples illustrate the present disclosure without, however, limiting it:

### Examples

1. In Vivo Test for Antiperspirant Activity

To determine the antiperspirant effect, an antiperspirant study was performed on the backs of 16 test persons. A 5.0% by weight aqueous solution of a phosphonate compound of the Formula (I) with a=b=1, R=*—(CH₂)ₓ—PO₃X⁺Y⁺ group where x=1 and X⁺=Y⁺=hydrogen or Na⁺ was prepared for this purpose.

75 μL of the prepared solution were applied on the back of 16 subjects on one side next to the spine. After 5 minutes, the treated sites were covered with occlusive non-adsorbent film. These non-adsorbent pads were removed after 2 hours. The compositions were each applied to the backs of the subjects for four consecutive days in the manner previously described. 24 h after the last application of the composition, absorbent pads were applied to the backs of the subjects at the sites where the compositions were previously applied. Furthermore, pads, which served as a control, were also applied on the other side of the spine at the same height. After the subjects had sweat for about 15 minutes at 80° C. in the sauna, the amount of perspiration absorbed by the pads was determined gravimetrically, wherein each composition was compared with the corresponding untreated site on the back. The sweat reduction was determined from the gravimetric determination of the amount of sweat, wherein all determined values were statistically significant.

A significant sweat reduction of 10.1% was determined using the aqueous solution of the phosphonate compound of Formula (I). The use of cosmetic agents containing specific phosphonate compounds, with the application of said agents to the skin, results in a reduction in transpiration and reduced body odor.

2. Formulations

The phosphonate compound a) used in the following examples is preferably a compound of the Formula (I) and/or of the Formula (II), in particular aminotrimethylene phosphonate (ATMP).

Antiperspirant cosmetic agents used as contemplated herein in the form of a solid stick (amounts specified in % by weight)

| | 1 | 2 | 3 | 4 | 5 | 6 |
|---|---|---|---|---|---|---|
| Stearyl Alcohol | 24.0 | 24.0 | 24.0 | 18 | 18 | 18 |
| Novata AB | — | — | — | 4 | 4 | 4 |
| Phosphonate compound a) | 22.0 | 30.0 | 22.0 | 17.6 | 17.6 | 17.6 |
| PPG-14 butyl ether | 10.0 | 10.0 | 10.0 | 15.3 | 15.3 | 15.3 |
| Hardened castor oil | 3.0 | 3.0 | 3.0 | 1.5 | 1.5 | 1.5 |
| (for example Cutina HR) | | | | | | |
| Myristyl myristate | 1.5 | 1.5 | 1.5 | — | — | — |
| DL-menthol | 0.2 | 0.2 | 0.2 | — | — | — |
| Eucalyptol | 0.2 | 0.2 | 0.2 | — | — | — |
| Anethole | 0.2 | 0.2 | 0.2 | — | — | — |
| Silica dimethyl silylates | 1.4 | 1.4 | 1.4 | — | — | — |
| Silica | 0.3 | 0.3 | 0.3 | — | — | — |
| Talc | — | — | — | 3 | 3 | 3 |
| Emulgin B1 | — | — | — | 3 | 3 | 3 |
| Perfume | 2.0 | 2.0 | 2.0 | 1 | 1 | 1 |
| Cyclomethicone | ad 100 | ad 100 | ad 100 | ad 100 | ad 100 | ad 100 |
| (at least 95% by weight | | | | | | |
| cyclopentasiloxane) | | | | | | |

Antiperspirant cosmetic agents used as contemplated herein in the form of a solid oil-in-water emulsion (amounts specified in % by weight)

| | 7 | 8 |
|---|---|---|
| Cutina ® AGS | 2.5 | 2.5 |
| Cutina ® FS45 | 3.5 | 3.5 |
| Eumulgin ® B2 | 0.8 | 0.8 |
| Eumulgin ® B3 | 0.8 | 0.8 |
| Diisopropyl adipate | 6.0 | 6.0 |
| Novata ® AB | 4.0 | 4.0 |
| Cutina ® CP | 5.0 | 5.0 |
| Cutina ® HR | 4.0 | 4.0 |
| Kester wax K62 | 5.0 | 5.0 |
| Phosphonate compound a) | 10 | 40 |
| Talc Pharma G | 10 | 10 |
| Perfume | 1.2 | 1.2 |
| 2-benzylheptan-1-ol | — | 0.3 |
| Sensiva SC 50 | 0.6 | 0.6 |
| 1,2-propanediol | 10 | 10 |
| Water, demineralized | ad 100 | ad 100 |

Antiperspirant cosmetic agents used as contemplated herein in the form of a liquid, sprayable micro-emulsion (specified in % by weight)

| | 9 | 10 | 11 | 12 |
|---|---|---|---|---|
| Plantaren ® 1200 | 1.7 | 1.7 | — | — |
| Plantaren ® 2000 | 1.1 | 1.4 | 2.4 | 2.4 |
| Glycerol monooleate | 0.71 | 0.71 | — | — |
| Dioctyl ether | 4.0 | 4.0 | 0.090 | 0.090 |
| Octyldodecanol | 1.0 | 1.0 | 0.020 | 0.020 |
| Perfume oil | 1.0 | 1.0 | 1.0 | 1.0 |
| Phosphonate compound a) | 20.0 | 10.0 | 5.0 | 5.0 |
| 1,2-propylene glycol | 5.0 | 5.0 | — | — |
| Glycerol | — | — | 5.0 | 5.0 |
| 2-benzylheptan-1-ol | 0.50 | — | — | — |
| Triethylcitrate | — | 0.50 | 0.50 | 0.50 |
| Water | ad 100 | ad 100 | ad 100 | ad 100 |

Antiperspirant cosmetic agents used as contemplated herein in the form of roll-ons (amounts specified in % by weight)

| | 13 | 14 | 15 | 16 |
|---|---|---|---|---|
| Ethanol 96% (DEP denatured) | 30 | 30 | 28 | 28 |
| Mergital ® CS 11 | 2.0 | 2.0 | — | — |
| Eumulgin ® B3 | 2.0 | 2.0 | 2.0 | 2.0 |
| Emulgin ® B1 | — | — | 2.0 | 2.0 |
| Phosphonate compound a) | 40 | 15 | 10 | 5.0 |
| Hydroxyethyl cellulose | 0.50 | 0.50 | 0.30 | 0.30 |
| EDTA | — | — | — | 0.050 |
| Cocamidopropyl PG-Dimonium | 0.20 | — | — | — |
| Chloride Phosphate | | | | |
| Perfume oil | 0.80 | 0.80 | 1.0 | 1.0 |
| Water | ad 100 | ad 100 | ad 100 | ad 100 |

Antiperspirant cosmetic agents used as contemplated herein in the form of a solid water-in-oil emulsion (amounts specified in % by weight)

| | 21 | 22 |
|---|---|---|
| Phosphonate compound a) | 15 | 30 |
| 1,2-propylene glycol | 13.0 | 13.0 |
| Cyclohexasiloxane | 6.00 | 6.00 |
| Finsolv TN | 8.00 | 8.00 |
| Abil EM 90 | 1.20 | 1.20 |
| Polyethylene wax (MW = 500 g/mol, | 10.0 | 10.0 |
| mp = 83 to 91° C.) | | |
| Polyalphaolefin wax (MW = 1800 g/mol, | 0.100 | 0.100 |
| mp = 41° C.) | | |
| EDTA | — | 0.0500 |
| Water | Ad 100 | Ad 100 |
| Perfume | 1.00 | 1.00 |

Antiperspirant cosmetic agents used as contemplated herein in the form of a gel application (amounts specified in % by weight)

| | 23 | 24 |
|---|---|---|
| Cyclopentasiloxane | 14.0 | 14.0 |
| Abil EM 97 | 3.00 | 3.00 |
| Ethanol 96% | 10.0 | 10.0 |
| Phosphonate compound a) | 30.0 | 10.0 |
| 1,2-propylene glycol | 20.3 | 20.3 |
| Water | ad 100 | ad 100 |
| EDTA | — | 0.0750 |
| Perfume | 1.00 | 1.00 |

Antiperspirant cosmetic agents used as contemplated herein (amounts specified in % by weight, based on the total weight of the propellant-free composition)

| | 27 | 28 | 29 | 30 |
|---|---|---|---|---|
| Phosphonate compound a) | 28.6 | 10.0 | 32.11 | 28.57 |
| Bentone 38 V CG | 5.00 | 3.93 | 4.00 | 5.00 |
| Propylenecarbonate | 1.50 | 0.71 | 1.50 | 1.80 |
| Fragrance | 7.14 | 6.50 | 5.00 | 6.50 |
| 2-ethylhexylpalmitate | — | 73.57 | — | — |
| Cyclomethicone D5 | Ad 100 | Ad 100 | Ad 100 | Ad 100 |
| Isopropyl myristate | 7.37 | — | 10.00 | 12.0 |
| Triethylcitrate | — | — | 10.5 | 12.0 |
| Dimethicone 2-10 cst | — | — | — | 10.0 |
| C₁₀-C₁₃ isoalkanes | — | — | 35.39 | — |

The example compositions 27 to 30 were filled into an aluminum spray can, optionally epoxy-phenolic coated, in a weight ratio of propellant (butane/propane/isobutane mixture) to suspension of 80:20 or 85:15 or 60:40 or 90:10.

Antiperspirant cosmetic agents used as contemplated herein (amounts specified in % by weight, based on the total weight of the propellant-free composition)

| | 31 | 32 | 33 |
|---|---|---|---|
| Phosphonate compound a) | 15.0 | 10.0 | 30.0 |
| C₁₀-C₁₃ isoalkane | 8.90 | 8.90 | 8.90 |
| Dow Corning ES-5227 DM | 1.40 | 1.40 | 1.40 |
| Isoceteth-20 | 0.500 | 0.500 | — |
| Dimethicone | 4.20 | 4.20 | 4.20 |
| Isopropyl myristate | 9.00 | 9.00 | 9.00 |
| 1,2-propanediol | 7.00 | 25.0 | 25.0 |
| Phenoxyethanol | 0.500 | 0.500 | 0.500 |
| Perfume | 2.50 | 2.50 | 2.50 |
| L-menthol | 0.400 | 0.300 | — |
| Trans-anethole | — | 0.300 | — |
| Eucalyptol | — | 0.300 | — |
| Perfume | 0.5 | 1.0 | 5.0 |
| Water | ad 100 | ad 100 | ad 100 |

The example compositions 31 to 33 were filled into an aluminum spray can, optionally epoxy-phenolic coated, in a weight ratio of propellant (butane/propane/isobutane mixture) to suspension of 80:20 or 85:15 or 60:40 or 90:10.

Antiperspirant cosmetic agents used as contemplated herein in the form of O/W emulsions as roll-on (amounts specified in weight)

| | 34 | 35 | 36 |
|---|---|---|---|
| Phosphonate compound a) | 40.0 | 13.0 | 13.0 |
| Talc | 1.0 | — | — |
| Bentonite | 3.0 | — | — |
| Hectorites | — | — | 5.00 |
| Brij S 2 | 2.50 | 2.50 | 2.50 |
| Brij S 721 | 1.50 | 1.50 | 1.50 |
| Perfume | 1.10 | 1.10 | 1.10 |
| Arlamol E | 0.500 | 0.500 | 0.500 |
| Bisabolol | 0.100 | — | — |
| Dry Flo PC | 0.100 | — | 0.100 |
| Dow Corning 2501 Cosmetic Wax | 0.100 | — | 0.100 |
| Tocopheryl acetate | 0.100 | 0.100 | 0.100 |
| Water | ad 100 | ad 100 | ad 100 |

Antiperspirant cosmetic agents used as contemplated herein (amounts specified in % by weight, based on the total weight of the propellant-free composition)

| | 37 | 38 | 39 | 40 |
|---|---|---|---|---|
| Phosphonate compound a) | 33.0 | 10.0 | 15.0 | 33.0 |
| Cyclomethicone | 12.0 | 9.40 | — | — |
| C₁₀-C₁₃ isoalkane | — | — | 9.40 | 8.90 |
| Dow Corning ES-5227 DM | — | 1.40 | 1.40 | 1.40 |
| Abil EM 90 | 3.00 | — | — | — |
| Brij IC 20 | — | — | — | 0.500 |
| Dimethicone | — | 4.20 | 4.20 | 4.20 |
| Isopropyl myristate | 9.00 | 9.00 | 9.00 | 9.00 |
| 1,2-propanediol | 7.00 | 7.00 | 7.00 | 7.00 |
| Phenoxyethanol | 0.500 | 0.500 | 0.500 | 0.500 |
| Perfume | 2.50 | 2.50 | 2.50 | 2.50 |
| Water | add 100 | add 100 | add 100 | add 100 |

The example compositions 37 to 40 were filled into an aluminum spray can, optionally epoxy-phenolic coated, in a weight ratio of propellant (butane/propane/isobutane mixture) to suspension of 80:20 or 85:15 or 60:40 or 90:10.

Antiperspirant cosmetic agents used as contemplated herein in the form of W/O emulsions (amounts specified in % by weight)

| | 41 | 42 | 43 | 44 |
|---|---|---|---|---|
| Phosphonate compound a) | 15 | 20 | 10 | 15 |
| Propylene glycol | 5.00 | 5.00 | 7.50 | 9.50 |
| C₁₂-C₁₅ alkyl benzoate | 8.04 | 8.04 | 8.04 | 8.04 |
| Dimethicone 2 cst | 6.43 | 6.43 | 6.43 | 6.43 |
| Dimethicone 5 cst | 1.57 | 1.57 | 1.57 | 1.57 |
| Polyethylene | 10.2 | 11.7 | 9.70 | 12.2 |
| Abil EM 90 | 0.998 | 0.998 | 0.998 | 0.998 |
| Abil EM 97 | 1.22 | 1.22 | 1.22 | 1.22 |
| Synthetic wax | 0.100 | 0.100 | 0.100 | 0.100 |
| Perfume | 1.50 | 1.50 | 1.20 | 1.50 |
| Water demin. | Ad 100 | Ad 100 | Ad 100 | Ad 100 |

Antiperspirant cosmetic agents used as contemplated herein in the form of anhydrous suspension roll-ons (amounts specified in % by weight)

| | 45 | 46 | 47 | 48 |
|---|---|---|---|---|
| Bentone 38 V (hydrophobic) | 2.0 | 3.0 | — | — |
| Propylenecarbonate | 1.50 | 2.0 | — | — |
| Ethylhexylpalmitate | 20.0 | 10.0 | — | — |
| Finsolv TN | — | 10.0 | 20.0 | 10.0 |
| Aerosil 972 V (hydrophobic) | — | — | 3.0 | 1.0 |
| Aerosil 200 | — | — | — | 1.0 |
| Protectate MOD 3 | — | 0.5 | — | 0.3 |
| Phosphonate compound a) | 40.0 | 15.0 | 30.0 | 10.0 |
| Perfume | 1.0 | 1.0 | 2.0 | 2.0 |
| Cyclopentasiloxane | Ad 100 | Ad 100 | Ad 100 | Ad 100 |

Antiperspirant cosmetic agents used as contemplated herein in the form of PIT pump atomizer emulsions (amounts specified in % by weight)

| | 49 | 50 | 51 | 52 |
|---|---|---|---|---|
| Eumulgin BA 10 | 3.8 | 3.5 | — | — |
| Cetiol PGL | 5.0 | 4.5 | — | — |
| Cetiol OE | 10 | 10 | — | — |
| Glycerol 86% | 5.0 | 5.0 | — | — |
| Eumulgade SE | — | — | 4.5 | 5.0 |
| Eumulgin B2 | — | — | 1.5 | 2.0 |
| Cetiol S | — | — | 15 | 15 |
| Myritol 318 | — | — | 5.0 | 5.0 |
| Propylene glycol 1,2 | — | — | 5.0 | 5.0 |
| Panthenol | — | — | 0.2 | 0.5 |
| Perfume | 1.0 | 1.0 | 1.0 | 1.0 |
| Phosphonate compound a) | 10 | 20 | 15 | 5.0 |
| Water | Ad 100 | Ad 100 | Ad 100 | Ad 100 |

Antiperspirant cosmetic agents used as contemplated herein in the form of a micro-emulsion (amounts specified in % by weight)

| | 53 | 54 | 55 | 56 |
|---|---|---|---|---|
| Plantaren 1200 | 1.7 | 1.8 | 2.0 | 1.7 |
| Plantaren 2000 | 1.2 | 1.4 | 2.4 | 1.2 |
| Glycerol monooleate | 0.7 | 0.7 | — | 0.7 |
| Dioctyl ether | 4.0 | 4.0 | 0.1 | 4.0 |
| Octyldodecanol | 1.0 | 1.0 | 0.1 | 1.0 |
| Perfume | 1.0 | 1.0 | 1.5 | 1.0 |
| Phosphonate compound a) | 5.0 | 10.0 | 15.0 | 20.0 |
| Propylene glycol 1,2 | 5.0 | 5.0 | — | 5.0 |
| Water | Ad 100 | Ad 100 | Ad 100 | Ad 100 |

The following commercial products were used:

| Commercial | | |
|---|---|---|
| product | INCI | Supplier/Manufacturer |
| Abil EM 90 | CETYL PEG/PPG-10/1 Dimethicone | Evonik |
| Abil EM 97 | Bis-PEG/PPG-14/14 Dimethicone, | Evonik |
| | Cyclomethicone | |
| Arlamol E | PPG-15 stearyl ether | Croda |
| Aerosil 200 | SILICA | Evonik |
| Aerosil 972 | SILICA DIMETHYL SILYLATE | Evonik |
| Bentone 38 V CG | Disteardimonium hectorite | Elementis |
| | | Specialties |
| Brij IC 20 | Isoceteth-20 | Croda |
| Brij S 2 | Steareth-2 | Croda |
| Brij S 721 | Steareth-21 | Croda |
| Cetiol PGL | HEXYLDECANOL, HEXYLDECYL | BASF |
| | LAURATE | |
| Cetiol OE | DICAPRYLYL ETHER | BASF |
| Cetiol S | DIETHYLHEXYLCYCLOHEXANE | BASF |
| Cutina ® AGS | GLYCOL DISTEARATE | BASF |
| Cutina ® CP | Cetyl Palmitate | BASF |
| Cutina ® FS45 | Palmitic Acid, Stearic Acid | BASF |
| Cutina ® HR | Hydrogenated Castor Oil | BASF |
| Dow Corning ® | Bis-PEG-18 methyl ether dimethyl silane | Dow Corning |
| 2501 | | |
| Dow Corning ES- | Dimethicone, PEG/PPG-18/18 dimethicone in | Dow Corning |
| 5227 DM | the weight ratio 3:1 | |
| Dry Flo PC | Aluminum Starch Octenylsuccinate | National Starch |
| Emulgade SE | Glyceryl Stearate, Ceteareth-20, Ceteareth-12, | BASF |
| | Cetearyl alcohol, Cetyl palmitate | |
| Eumulgin ® B1 | Ceteareth-12 | BASF |
| Eumulgin ® B2 | Ceteareth-20 | BASF |
| Eumulgin ® B3 | Ceteareth-30 | BASF |
| Eumulgin ® BA 10 | BEHENETH-10 | BASF |
| Kester wax K62 | Cetearyl behenate | Koster Keunen |
| Finsolv TN | C12-15 alkyl benzoates | Innospec |
| Mergital ® CS 11 | Ceteareth-11 | BASF |
| Myritol 318 | CAPRYLIC/CAPRIC TRIGLYCERIDE | BASF |
| Novata ® AB | Cocoglycerides (mp 30-32° C.) | BASF |
| Plantaren ® 1200 | LAURYL GLUCOSIDE, approx. 50% AS | BASF |
| Plantaren ® 2000 | DECYL GLUCOSIDE, approx. 50% AS | BASF |
| Protectate MOD 3 | 2-BENZYLHEPTANOL, | Symrise |
| | PHENOXYETHANOL, HELIOTROPINE | |
| Sensiva ® SC 50 | 2--ethylhexylglycerol ether | Schülke & Mayr |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic method for reducing transpiration of the body and/or reducing body odor triggered by the transpiration, in which an antiperspirant cosmetic is applied to the human skin and remains at the application site for at least about 1 hour, the antiperspirant cosmetic comprising, in a cosmetically compatible carrier, based on the total weight of the cosmetic agent:
a) at least one phosphonate compound comprising at least one tertiary amine group and/or at least one secondary amine group and/or at least one primary amine group and/or at least one hydroxyl group, and
b) at most about 1% by weight of antiperspirant aluminum and/or aluminum zirconium salts.

2. The method according to claim 1, wherein the cosmetic agent comprises 0% by weight of antiperspirant aluminum and/or aluminum zirconium salts, based on the total weight of the cosmetic agent.

3. The method according to claim 1, wherein the at least one phosphonate compound, which comprises at least one tertiary amine group or at least one secondary amine group, has the Formula (I) 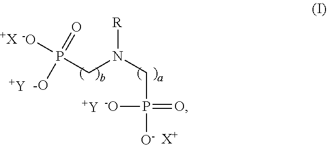
wherein
R stands for hydrogen, a *—(CH₂)ₓ—PO₃X⁺Y⁺ group with x=0 to 4, a *—[CH₂—CH₂—NR¹]y-CH₂—CH₂—N(R¹)₂ group with y=0 to 10 and R¹=*—CH₂—PO₃X⁺Y⁺ or a *—CH₂—CH₂—[NR²—CH₂—CH₂]_{z}—N(R³)₂ with z=1 to 10, R²=*—(CH₂—CH₂—O)ₒ—H with o=1 to 30 and R³=*—CH₂—PO₃X⁺Y⁺,
a and b, each independently of one another, stand for integers from 0 to about 10,
X⁺ and Y⁺, each independently of one another, stand for a mono- or polyvalent cation chosen from the group of alkali metals, alkaline earth metals, transition metals or organic cations, ammonium or hydrogen.

4. The method according to claim 3, wherein in the Formula (I), a and b each stand for the integer 1, R stands for a *—(CH₂)ₓ—PO₃X⁺Y⁺ group with x=1 and X⁺ and Y⁺, each independently of one another, stand for hydrogen or Na⁺.

5. A method according to claim 3, wherein in the Formula (I), a and b each stand for the integer 1, R stands for a *—[CH₂—CH₂—NR¹]y-CH₂—CH₂—N(R¹)₂ group with y=0 and R¹=*—CH₂—PO₃X⁺Y⁺ and X⁺ and Y⁺, each independently of one another, stand for hydrogen or Na⁺.

6. The method according to claim 1, wherein the at least one phosphonate compound which comprises at least one hydroxyl group, has the Formula (II) 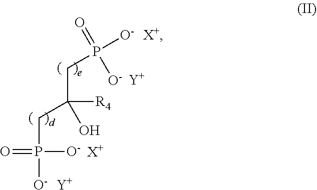
wherein
R₄ stands for a linear or branched, saturated or unsaturated C₁-C₃₀ alkyl group,
d and e, each independently of one another, stand for integers from 0 to about 10,
X⁺ and Y⁺, each independently of one another, stand for a mono- or polyvalent cation from the group of alkali metals, alkaline earth metals, transition metals or organic cations, ammonium or hydrogen.

7. The method according to claim 6, wherein in the Formula (II), R₄ stands for a C₁₁ alkyl group, d and e each stand for the integer 0 and X⁺ and Y⁺, each independently of one another, stand for hydrogen or Na⁺.

8. The method according to claim 1, wherein the cosmetic agent comprises the at least one phosphonate compound a) in a total amount of from about 0.1 to about 40% by weight, based on the total weight of the cosmetic agent (M1).

9. A packaging unit comprising, assembled separately from one another,
a) at least one first container comprising a cosmetic agent comprising, in a cosmetically compatible carrier, at least one phosphonate compound comprising at least one tertiary amine group and/or at least one secondary amine group and/or at least one primary amine group and/or at least one hydroxyl group and at most about 1% by weight of aluminum and/or aluminum zirconium antiperspirant salts based on the total weight of the cosmetic agent, and
b) at least one second container comprising a cosmetic agent comprising at least one antiperspirant active substance.

10. (canceled)

11. The method according to claim 1, wherein the least one phosphonate compound comprises at least one hydroxyl group.

12. The method according to claim 3, wherein a and b, each independently of one another, stand for integers 0 or 1.

13. The method according to claim 6, wherein d and e, each independently of one another, stand for integers 0 or 1.

14. The method according to claim 6, wherein d and e are each 0.

15. The method according to claim 1, wherein the cosmetic agent comprises the at least one phosphonate compound a) in a total amount of from about 2.0 to about 10.0% by weight, based on the total weight of the cosmetic agent (M1).

16. The method according to claim 1, wherein the least one phosphonate compound comprises aminotrimethylene phosphonate.
